SEQUENCE LISTING (17 sequences)

SEQ ID NO: 1 - protein, 10 aa, Homo sapiens
  Autonomous activate region Subset 2/1
KLTVLRQPKA

SEQ ID NO: 2 - protein, 8 aa, Homo sapiens
  Autonomous activate region Subset 2/2
VAPGKTAR

SEQ ID NO: 3 - protein, 10 aa, Homo sapiens
  Autonomous activate region Subset 4/1
PTIRRYYYYG

SEQ ID NO: 4 - protein, 9 aa, Homo sapiens
  Autonomous activate region Subset 4/2
NHKPSNTKV

SEQ ID NO: 5 - protein, 116 aa, Homo sapiens
  Autonomous activate region Subset 2 VH
EVQLVESGGGLVKPGGSLRLSCAASGFTFRSYSMNWVRQAPGKGLEWVSSIISSSSYIYYADSVKGRFTISRDNAKNSLYLQMNSLRAEDTALYYCARDQNAMDVWGQGTTVTVSS

SEQ ID NO: 6 - protein, 215 aa, Homo sapiens
  Autonomous activate region Subset 2 LC
SYELTQPPSVSVAPGKTARITCAGNNIGSKSVHWYQQKPGQAPVLVIYYDSDRPSGIPERFSGSNSGNTATLTISRVEAGDEADYYCQVWDSGSDHPWVFGGGTKLTVLRQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 7 - protein, 224 aa, Homo sapiens
  Autonomous activate region Subset 4 HC
QVQLQQWGAGLLKPSETLSLTCAVYGGSFSGYYWTWIRQSPGKGLEWIGEINHSGSTTYNPSLKSRVTISVDTSKNQFSLKLNSVTAADTAVYYCARGYGDTPTIRRYYYYGMDVWGQGTTVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKC

SEQ ID NO: 8 - protein, 220 aa, Homo sapiens
  Autonomous activate region Subset 4 LC
DIVMTQSPLSLPVTLGQPASISCRSSQSLVHSDGNTYLNWFQQRPGQSPRRLIYKVSDRDSGVPDRFSGSGSGTDFTLKISRVEAEDVGLYYCMQGTHWPPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 119 aa, Mus musculus
  AVA-mAb01 HC
QVQLQQSGPGLVQPSQSLSITCTVSGFSLTSYGIHWVRQSPGKGLEWLGVIWRGGGTDSNAAFMSRLSITKDNSKSQVFFKMNSLQADDTAIYYCARSRYDEEESMNYWGQGTSVTVSS

SEQ ID NO: 10 - protein, 107 aa, Mus musculus
  AVA-mAb01 LC
QIVLTQSPASLSASVGETVTITCRASGNIHSYLAWYQQKQGKSPQLLVYNAKTLADGVPSRFSGSGSGTQYSLKINSLQPEDFGSYYCQHFWNTPPTFGAGTKLELK

SEQ ID NO: 11 - protein, 8 aa, Mus musculus
  AVA-mAb01 CDR1 HC
GFSLTSYG

SEQ ID NO: 12 - protein, 7 aa, Mus musculus
  AVA-mAb01 CDR2 HC
IWRGGGT

SEQ ID NO: 13 - protein, 13 aa, Mus musculus
  AVA-mAb01 CDR3 HC
ARSRYDEEESMNY

SEQ ID NO: 14 - protein, 6 aa, Mus musculus
  AVA-mAb01 CDR1 LC
GNIHSY

SEQ ID NO: 15 - protein, 4 aa, Mus musculus
  AVA-mAb01 CDR2 LC
NAKT

SEQ ID NO: 16 - protein, 9 aa, Mus musculus
  AVA-mAb01 CDR3 LC
QHFWNTPPT

SEQ ID NO: 17 - protein, 8 aa, Mus musculus
  Autonomous activate region Subset 4/2
VSSASTKG

(19) United States
(12) Patent Application Publication
(10) Pub. No.: US 2022/0356244 A1
(43) Pub. Date: Nov. 10, 2022

(54) METHOD FOR SELECTING BIOLOGICAL BINDING MOLECULES

(71) Applicant: AVA Lifescience GmbH, Denzlingen (DE)

(72) Inventors: Holger KLAPPROTH, Freiburg i. Br. (DE); Marc A. KESSEMEIER, Emmendingen (DE); Ulrich BIRSNER, Freiburg i. Br. (DE)

(21) Appl. No.: 17/607,985

(22) PCT Filed: May 2, 2019
(86) PCT No.: PCT/EP2019/061302

§ 371 Date: Jul. 6, 2022

(51) Int. Cl. C07K 16/28; G01N 33/569; C07K 16/30

(52) U.S. Cl. CPC C07K 16/2803; G01N 33/56972; C07K 16/3061; C07K 2317/14; C07K 2317/24; C07K 2317/52

(57) ABSTRACT

The present invention relates to the field of producing, identifying, and selecting biological binding molecules, e.g. in particular antibodies or fragments thereof, which selectively bind to autonomously active B-cell receptors or B-cell receptor complexes. The method is used in order to select a biological binding molecule which specifically binds to an autonomously active or autonomously activated B-cell receptor as the target receptor, but not to an inactive or non-activated B-cell receptor, and is carried out in a cell-based system using immature B cells which are in the pro/pre-stage and cause a ‘triple knockout’ of the genes for RAG2 or RAG1, lambda5, and SLP65.

The instant application contains a Sequence Listing, which has been submitted electronically in ASCII format and is hereby incorporated by reference in its entirety. Said ASCII copy, created on Jul. 6, 2022, is named 17607985_Substitute_Sequence_Listing_6Jul2022 and is 11,209 bytes in size.

The present invention relates to the field of the production, identification and selection of biological binding molecules such as, in particular, antibodies or fragments thereof, which selectively bind to activated B-cell receptors or B-cell receptor complexes.

In biochemistry, a protein or protein complex is referred to as a receptor (from the Latin ‘recipere,’ ‘receive’) when signal molecules that are able to trigger signal processes in the cell interior through the binding event can bind to it. A receptor can receive signals from the outside and be located on the surface of a biomembrane, or it can be verifiable in the cytosol of the cell. Receptors possess a specific binding site for their physiological agonists or antagonists (binding partner, ligand).

Membrane receptors are located on the surface of biomembranes and consist of proteins which are frequently provided with modifications (e.g., carbohydrate chains). They possess a specific fit for small molecules, so-called ligands, or for parts of larger molecules which bind to the receptor structure by complementing them as a complementary structure (simplistically referred to as the ‘key-lock principle’).

Therefore, receptors can serve the purpose of receiving and forwarding signals (signal transduction), or be functionally involved in the binding of cells (cell adhesion), or in the transport of substances in the cell (membrane transport). Furthermore, they can offer virions the possibility of docking to the appropriate host cell and infecting it.

Among the membrane receptors important for cell contacts are both cell adhesion molecules, which facilitate cell-cell contact such as cadherins, selectins and immunoglobulins, and also those that are involved in the formation of cell-matrix contacts and anchor cells on the extracellular matrix, such as integrins.

Membrane receptors do not only occur on the plasma membrane, but also on biomembranes of the organelles in the cell interior. While external cell membrane receptors relate the cell to the exterior as their surroundings, individual organelles in the interior of the cell are related via receptors to the cytoplasm, cytoskeleton or to one another.

Receptors in the cell membrane are divided into ionotropic and metabotropic receptors, depending on their effect.

Ionotropic receptors constitute ion channels which, in binding a suitable ligand, open with a higher probability and thus alter the conductivity of the membrane.

However, metabotropic receptors do not form channels or pores, but, in binding their ligand, activate a downstream “second messenger” (e.g., a G protein or a protein kinase) and thus modulate intracellular signaling cascades by concentration changes of secondary messengers, which, however, can indirectly also result in a modification in the membrane permeability.

For many receptors, natural ligands exist, which lead to the activation of these receptors and trigger a ‘second messenger’ cascade. Along with natural ligands, there are also substances which bind to the receptor and either activate it (agonists) or inactivate it (antagonists). Examples of receptor agonists are e.g., antigens including allergens, opioids, nicotine, salbutamol, muscarine, cytokines, and neurotransmitters.

The B-cell receptor or the B-cell receptor complex (BCR) constitutes a special receptor in this respect. This BCR is expressed by B cells and constitutes virtually a membrane-bound antibody. The BCR is formed in great variety in maturing B cells.

The development of the B cells takes place in humans and also in some other mammals in the bone marrow or in the fetal liver. The signals that are necessary for the development program receive the developing lymphocytes of so-called stromal cells. In B cell development, the formation of a functioning B-cell receptor (the membrane-bound form of the ‘antibody’) is of critical importance. Only with this antigen receptor are mature B cells later able to detect foreign antigens and bind them to hostile structures by forming corresponding antibodies. The antigen specificity of the receptor is determined by the linking of specific gene segments. The segments are called V, D and J segments, which is why the process is referred to as V(D)J recombination. In this case, these segments, which form the antigen-binding part of the B-cell receptor, are rearranged. The entire receptor consists of two identical light protein chains and two identical heavy protein chains, wherein the heavy chains are in each case linked to the light chains via disulfide bridges. In the VDJ recombination, the V, D and J segments of the heavy chain of the B-cell receptor are first linked, followed by the V and J segments of the light receptor chain. Only when the genes are successfully rearranged, which is referred to as productive gene rearrangement, can the cell transition to the respective next development step.

B cells, which react to endogenous antigens during their maturation in the bone marrow, die off in the majority of cases due to apoptosis. Small quantities of autoreactive cells, among others against thyroglobulin or also collagen, can be traced in the blood of healthy humans (Abul K. Abbas: Diseases of Immunity in Vinay Kumar, Abul K. Abbas, Nelson Fausto: Robbins and Cotran—Pathologic Basis of Disease. 7th Edition. Philadelphia 2005, p. 224 et seqq.).

If antibodies are generated against receptors, animals are, as a rule, immunized with the receptor (purified, cloned, or as peptide fragments) and hybridoma cells are generated. These hybridoma cells produce antibodies which are then tested by means of ELISA or by means of expressed receptors in cell systems. For this purpose, conventionally established cell lines are used, since only they can be easily cultivated. In the process, antibodies can be generated, which bind relatively specifically to a specific receptor type (e.g., anti-IgG1, anti-IgE). However, this frequently results in cross reactions with other receptors or other epitopes.

For a therapeutic application of BCR antibodies, it is usually not sufficient to use only one antibody against the BCR in general, since such a broadband use can trigger considerable side effects. Instead, it would be desirable to provide an antibody that selectively binds to a receptor that has a (pathophysiological) activation, in particular an autonomous activation. Such an antibody is not known in the prior art and a method for its production or isolation through selection does not exist.

Therefore, the problem addressed by the invention is that of providing a system for producing or isolating and identifying or selecting biological binding molecules, in particular antibodies or functional fragments thereof, which selectively bind to BCRs, which are autonomously activated or are in an autonomously activated state. For this purpose, it is important that the receptors or receptor complexes used for the selection have a correct folding and are thus functional.

The problem is solved by the provision of a method according to the main claim. Preferred embodiments are the subject matter of corresponding dependent claims.

Before going into detail about the individual aspects of the present invention, there will be a clarification of relevant terms used within the scope of the present description.

“Biological binding molecules” presently refer, for example but not exclusively, to antibodies including fusion proteins. Advantageously and therefore preferably, such an antibody is selected from the group consisting of an IgG antibody, an IgM antibody, a humanized IgG antibody, and a human antibody, into which the detection sequence of the epitope is inserted. Such a binding molecule can also be provided in the form of a functional fragment of the entire antibody, e.g., as a fab fragment. A binding molecule can also comprise further regions that, e.g., result in the killing/dying of neoplasias and thus have the functionality of an immunotoxin and/or an immunocytokine. In particular, such a binding molecule can also be membranous or cell-bound. Such a membranous form of a binding molecule is, e.g., the chimeric antigen receptor (CAR) on CAR-T cells. For diagnostic applications, the binding molecule can comprise verifiable markings, in particular one or more fluorescent dyes.

It is the task of the B-cell receptor complex (BCR) on the surface of a B cell to detect and bind pathogens. As already mentioned, this binding leads to a conformational change of the BCR, as a result of which a signal cascade is triggered, which ultimately leads to an activation of the B cell. Since the process of generating such a BCR is based on a random congregation of gene segments, it is possible that the newly formed BCR detects undesirable endogenous structures and is thus “permanently activated.” In order to prevent the development of such a “permanently active or activated” BCR, different endogenous protective mechanisms exist. However, if they are overcome due to a pathological change of the developing B cell, a malignant or also autoimmune manifesting illness can develop therefrom.

An “autonomously active” or “autonomously activated” BCR, however, is a special type of a permanently active BCR. While the conventional activation proceeds from an external antigen (see above), the autonomously active BCR results from its interaction with membrane structures on the surface of the same cell. For the symptoms of chronic lymphatic leukemia (CLL), it was possible to show an interaction between BCRs triggering the autonomous activation, which were located adjacent to one another on the surface of the same cell (M. Dühren-von Minden et al.; Nature 2012). A further example of an autonomously active BCR is the pre-BCR which is expressed in the course of the development of a B cell as a development check. However, along with the interaction of adjacent receptors (BCR:BCR), an interaction between the receptor and a membrane protein (BCR:membrane protein) can also result in an autonomously active or activated BCR.

The solution of the problems according to the invention with respect to the provision of a biological binding molecule, in particular of an antibody or of a functional fragment thereof, with—compared to conventional antibodies—a selective specificity with respect to autonomously active or activated B-cell receptors or B-cell receptor complexes (BCRs) is based on the surprising discovery that B-cell receptors can be found on tumor cells of patients with chronic lymphatic leukemia (CLL), which are autonomously active, and that these autonomously active receptors are characterized by the presence of common epitopes which cannot be traced in the corresponding receptors of healthy cells of the same patients. These cells can thus be specifically detected and treated by means of an antibody on the basis of the presence of autonomously active B-cell receptors which are characterized by the occurrence of the above-mentioned epitopes, so that healthy B cells without this characteristic are not affected by this, as a result of which the treatment can be carried out more specifically and with fewer adverse effects.

Within the scope of the numerous experiments conducted for the present invention, it was surprisingly found, however, that antibodies cannot be produced and selected with particular specificity for these modified receptor regions (epitopes) using standard methods. Only after the experimental conditions were adapted such that, within the scope of binding studies, genetically altered cells were used whose modified B-cell receptors were in a native and activated state, was it possible to obtain suitable antibodies with the desired and required specificity. In other words, it is of essential importance for the solutions proposed according to the invention that the cells used in binding studies for the selection of suitable prophylactic or therapeutic and diagnostic antibodies present their modified regions (epitopes) in a largely native and activated form. It was found that so-called pro-/pre-B cells are particularly suitable due to their physiological constitution. The provision of such specific antibodies and functional fragments thereof, which likewise have this specific binding behavior, thus facilitates a tumor-specific treatment which is characterized by a significantly improved treatment success and, due to the reduction of undesirable systemic effects, a significantly increased therapeutic success. Within the scope of diagnostic applications, the possibility of using such specific antibodies means a much more accurate analysis with a much higher significance with respect to the evaluation of a state of a patient to be assessed.

As already mentioned, the present invention provides a method for producing (identifying and selecting) biological binding molecules in the form of antibodies or functional fragments thereof, wherein the binding molecules selectively bind to specific epitopes of autonomously active membranous receptors or receptor complexes of B cells.

Two variants (subset 2; subset 4) of the autonomously active BCR are known, which differ from one another with respect to their respective characterizing molecular motifs (epitopes) (Minici, C. et al., Distinct homotypic B-cell receptor interactions shape the outcome of chronic lymphocytic leukaemia, Nature Comm. (2017)). Both variants have differing short amino acid sequences which are each specific for these variants. It is known to a person skilled in the art that, in addition to the cited subsets, other B-cell receptors are also autonomously active. The region of subset 2 relevant for the autonomously active functionality of the receptor is in this case characterized by the amino acid sequences KLTVLRQPKA (SEQ ID NO. 1) and VAPGKTAR (SEQ ID NO. 2) of the light chain, while the region of subset 4 relevant for the autonomously active functionality of the receptor is defined by the amino acid sequences PTIRRYYYYG (SEQ ID NO. 3) and NHKPSNTKV (SEQ ID NO. 4) of the variable part of the heavy chain. The sequences for the subsets 2 and 4 used to generate the murine antibodies within the scope of immunization are specified in SEQ ID NOS. 5 and 6 (vHC; LC) or 7 and 8 (vHC; LC). For the sake of completeness, a further target sequence or a further epitope is specified in SEQ ID NO. 17 (VSSASTKG) with specificity for the variable part of the heavy chain of a BCR of the subset 4. Along with the target sequences (epitopes) responsible for the formation of the autonomously active state of the BCR (subset 4) according to SEQ ID NOS. 3 and 4, the sequence according to SEQ ID NO. 17 thus constitutes a further characteristic property for this subset.

It must be noted that the locating and characterizing of subsets 2 and 4 as two variants of the B-cell receptor in patients with critical progression of the disease is based on the analysis of numerous individual case studies and therefore does not mean that, in a possible plurality of other subtypes of the BCR, the same target sequences (epitopes) characterizing the two known subtypes are not present and correlate with a serious progression of the disease.

Although it should in principle be possible to generate antibodies against both of these subsets with standard methods, e.g., in mice, it was surprisingly observed that an immunization using peptides did not lead to the formation of the desired specific antibodies. The immunization using individual chains of the receptor, e.g., the use of the light chain of the BCR comprising the modified sequence regions, did also not bring the desired success, which is why mice were finally immunized with the recombinantly produced soluble form of the BCR (cf. SEQ ID NOS. 5 and 6). Subsequently, it was possible to obtain immune cells with the desired specificity from these mice and transform them into hybridoma cells through cell fusion. However, it was surprisingly found that the active antibodies could not be identified by means of the ELISA test or other standard methods. However, the clones identified as potential binding partners in a first step by means of ELISA proved to be either non-specifically binding or did not bind to the autonomously active receptor (including the SEQ ID NOS. 1 and 2) after the selection and therefore had to be discarded.

The methods applied up to this realization comprised not only standard methods such as ELISA and SPR but also the intracellular expression in fibroblasts with an intracellular FACS dye as binding control.

After an elaborate further series of tests, it could be shown that a successful selection of suitable binding molecules according to the invention can be carried out neither with free receptors or their fragments nor with membranous or intracellular receptor fragments. Instead, it was observed that the selection using only one cell system succeeded, within the scope of which the complete and functional B-cell receptor was presented in a membranous manner. In this case, it is of great importance that the BCR with its modified regions (epitopes) in or on these cells is autonomously actively present or presented. Only with this approach, whose conditions reflect a largely physiologically native in situ scenario, was it possible to identify an antibody which binds highly specifically and selectively only to the tumor cells, i.e., to B cells, which express a BCR with an epitope on their cell membrane, which is characteristic for the subset 2 or subset 4 of this cell type, but not to other B cells or their receptors (BCRs) which by definition do not constitute B cells of the subset 2 or 4. In other words, the discovered binding molecule according to the invention selectively binds to autonomously active B-cell receptors which are characterized by the presence of structural domains or epitopes (target sequences) and are the cause for the autonomously activated state of the B-cell receptors. The selective binding behavior of the binding molecule according to the invention means that it does not bind to receptors or other membrane structures of B cells with no structural domain or no epitope which are the cause for the autonomously active state of the B cells. Therefore, the binding molecule proposed for use according to the invention does not bind selectively to target sequences of the B-cell receptor, which are not characteristic for the subset 2 or the subset 4, and in particular does not bind to a B-cell receptor that has none of the sequences SEQ ID NOS. 1, 2, 3 and/or 4.

It could further be shown that, despite of the difficult handling and the time-consuming isolation, the use of arrested pro-/pre-B cells, which have been obtained from ‘triple knockout’ mice (TKO), are especially well-suited to express these receptors and, within the scope of a test system, to be used to identify these receptors. The stage of the pro-/pre-B cells is naturally designed to carry out the maturation and selection of the BCRs, and the cells of this stage are, due to their enzyme composition (chaperone, etc.), particularly suitable to correctly fold even “difficult” BCR components and present them on their surface in a sufficiently physiologically native form. The subsequently described deletions (knockouts) prevent a change in the desired BCR by means of a recombination or the use of the surrogate light chain. By using these cells or this cell type of arrested pro-/pre-B cells for the expression and presentation of BCRs within the scope of a selection of antibodies with selectively specific binding behavior with respect to autonomously active or activated B-cell receptors, a selection platform is provided, which, when compared to the conventional systems used for selection in the prior art, is characterized by a much higher quality, which justifies the high expense of using primary TKO cells or their cultivation over few passages.

As a special feature, these cells have the following knockouts in their genome:
the knockout of RAG2 prevents the somatic recombination of inherent heavy and light immunoglobulin chains, which is why the endogenous formation of a BCR is excluded. This leads to an arrest, a blocking or ‘freezing’ of correspondingly treated B cells in this developmental stage. It is known that RAG1 and RAG2 form a complex which makes the usual VDJ-rearrangement possible and which is why a knockout of RAG1 is an equally acting agent and thus represents an alternative to the knockout of RAG2 and is comprised by the teaching according to the invention.
the deletion of lambda5, a part of the surrogate light chain, prevents the formation of a pre-BCR. Since the pre-BCR is autonomously active, this would interfere with the detection of an autonomously active receptor. Since a new BCR is being cloned in the cell, a pre-BCR is undesirable because it would appear with the desired heavy chain (HC) in connection with the undesirable surrogate light chain on the surface and interfere with the selection.
the knockout of SLP65, an adapter protein of central importance in the BCR signal path, prevents the activation of the cell by a possibly reconstructed BCR.

The combination of the knockouts of RAG2 or RAG1 and lambda5 leads to a blockade in the transition from the pro-B cell stage to the pre-B cell stage, which classically is characterized by the beginning rearrangement of the VDJ segments of the heavy chain (HC). Therefore, these are pro-/pre-B cells.

The knockout of RAG2 or RAG1 and lambda5 is sufficient for the expression of the BCR and the selection of the suitable antibody. By means of the reconstitution with the inducible SLP65, it is possible to measure the activity of the BCR. Since autonomously active BCRs are selected, this step is a preferred embodiment of the invention.

In this case, the method of selection is the measurement of the Ca-flux after the induction of the SLP65 by means of FACS analysis and the use of a Ca²⁺-dependent dye such as Indo-1. These methods are known to a person skilled in the art (see M. Dühren-von Minden et al.; Nature 2012).

With the first two knockouts, it was ensured that only the “BCR of interest” is expressed on the surface. Through the use of an inducible SLP65, with which the cells were reconstituted, the function of the expressed BCRs can additionally be characterized and the autonomously active state of the BCRs on the surface can thus be verified prior to the selection.

After the previously described selection of suitable hybridoma cells, it was possible to isolate the antibodies suitable for diagnostic, prophylactic and/or therapeutic purposes in the form of monoclonal antibodies in larger quantities. By means of sequencing the DNA of these cells, it was possible to determine the binding site of the antibody (cf. SEQ ID NOS. 9 and 10). Corresponding methods are known to a person skilled in the art and are also commercially available. In this case, it is advantageous if a large number of hybridoma cells is isolated and those with the best binding activity (specificity and binding thickness/affinity) are selected.

By means of the thus obtained information about the binding site, the sequence coding for this purpose was inserted into an expression plasmid with the DNA of a human antibody sequence in order to produce a humanized monoclonal antibody with the desired specificity via the usual path of recombination. Due to their unique specificity, these humanized antibodies showed a better prophylactic and therapeutic efficacy at comparatively very few adverse effects when compared to conventional active agents. It is clear to a person skilled in the art that these humanized antibodies can be produced in large quantities using biotechnical methods. Standardized methods can be used to purify the synthesized antibodies, e.g., combinations of precipitation, filtration and chromatography, which is sufficiently well-known to a person skilled in the art, wherein it must be noted that the antibodies should not be denaturized and possible foreign substances such as proteins, pyrogens and toxins should be quantitatively removed.

Preferably, the desired antibodies are expressed in systems in which the antibody undergoes a glycosylation, in particular a human glycosylation. Such systems are sufficiently well-known to a person skilled in the art and include the use of insect cells (S2 cells), mammal cells (CHO cells) and, particularly preferably, human cells, for example, cells of the type HEK293T.

The sufficiently purified antibody can by itself be therapeutically effective, provided that it has an isotype that elicits a specific immune response, e.g., an IgG subtype, which leads via Fc receptors to an immune response against the tumor.

However, the antibody can also be present as a fragment. In this case, it is important that the antigen binding site is present in the fragment, i.e., it is a functional fragment. Such fragments can be produced, e.g., by means of protease treatment as F(ab) fragments. Since these fragments are truncated in the constant part of the antibody, it is in this case advantageous within the scope of prophylactic or therapeutic applications to insert an effector molecule to kill off neoplasias.

Individual aspects of the present invention will be described in greater detail on the basis of examples.

Before detailed descriptions about the experimental procedure are provided, reference is made to the following statements.

The production and identification of antibodies, which bind selectively to the modified B-cell receptors, were characterized by large and unanticipated problems. The hybridomas were generated by means of standard methods. The supernatants of the hybridoma groups were pooled and examined by means of ELISA for positive binding events (soluble B-cell receptors on the ELISA plate). Positive pools were isolated and the individual clones tested. In the process, no more positive clones were surprisingly identified in the ELISA. The positive ELISA signals of the pools eventually proved to be unspecific bindings.

In order to create better epitopes for the detection of the antibodies, the light chain of the BCR was expressed in fibroblasts. This was supposed to ensure the correct folding of the protein which carries the responsible motif (epitope) for the autonomous signal. Intracellular FACS analyses were carried out with these cells. No positive clone (antibody) could be identified.

For this reason, RAMOS cells (human Burkitt lymphoma cell line) were modified in a further experiment, so that they exhibited functionally modified BCRs. This was supposed to ensure the completely correct biosynthesis, folding and modification of the BCR. For this purpose, the cell's own BCR was deleted by means of CRISPR and the desired BCR was the molecular-biologically reconstituted (electroporation of CMV vectors). These cells were used for testing positive binding events. Once again, no positive clone could be verified by means of FACS.

However, the use of murine TKO (‘triple knock-out’) cells (arrested pro-/pre-B cells), introduced in the desired BCR by means of a gene shuttle, surprisingly yielded a positive clone, even though this was not ensured by the human cell system.

The expression of the BCR was determined by means of anti-IgM and anti-LC antibodies on the FACS. For this purpose, some cells were taken and each dyed with 5 μl antibody in a total volume of 100 μl in PBS.

With these cells as a “target,” it was possible to identify an antibody by means of FACS, which specifically binds to the modified region that causes and characterizes the permanent activation of the BCR, even though a binding to the same receptor type in RAMOS cells was not successful!

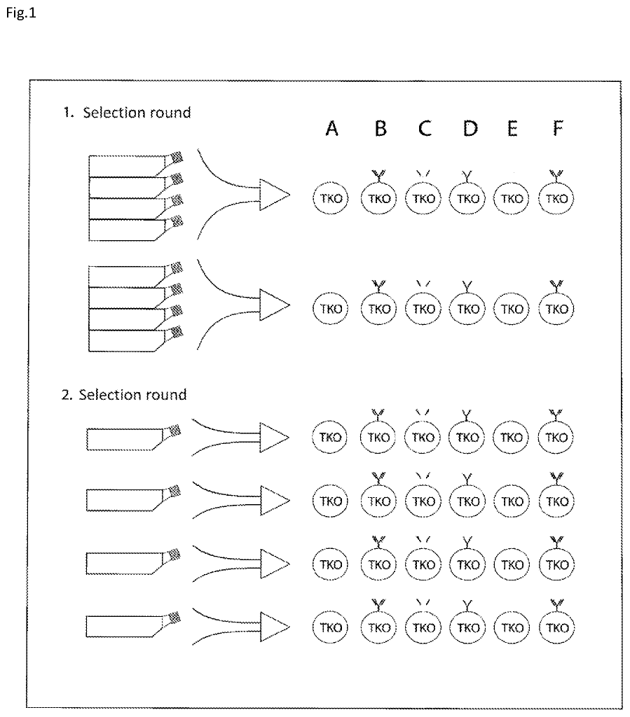

For this purpose, the cells, which carried the desired BCR on the surface, were first incubated with the pooled supernatants, and after a few washing steps, the bound antibodies were detected by means of secondary antibodies. For a specific selection, TKO cells (TKOs) were used, which expressed different versions of the desired BCR. The selection matrix shown in FIG. 1 is exemplary for the selection of a CLL subset 2 BCR and was used for the identification and selection of positive clones. For easier identification, the supernatants of the hybridomas were pooled and measured. The groups which showed a binding were isolated, and the supernatants of the respective hybridomas were tested for binding.

The confirmation that the selected antibody specifically binds to the modified BCR and not to other BCR variants took place by means of two blind samples, i.e., with cells without BCR (see FIG. 1A) and with cells with non-CLL BCR (see FIG. 1 E). Primary B cells from the blood of leukemia patients were analyzed for binding by means of FACS. The selected antibody was able to identify specifically those BCRs which had the target structure. This was confirmed at the genomic level. Samples without this target structure had no binding.

In the following, the invention will be described in greater detail using examples while taking FIG. 1 into account.

### EXAMPLE 1

For the production of triple knockout cells (TKO), transgene mice that have a respective knockout for the genes lambda5, RAG2 and SLP65 are the starting point (Dühren von Minden et al., 2012, Nature 489, p. 309-313). The preparation of such mice is known to a person skilled in the art and belongs to the prior art. For isolating the cells, the bone marrow of the femur of the mice was extracted after their death. The cells thus obtained were subsequently cultured under conditions that facilitate the survival of pro-/pre-B cells (37° C., 7.5% CO₂, Iscove's medium, 10% FCS, P/S, murine IL7). After several passages, a FACS sorting was carried out as a control, which sorts the pro-/pre-B cells and subsequently cultures them again. The markers used for this purpose are known to a person skilled in the art.

For the reconstitution with a ‘BCR of interest,’ the corresponding coding sequences for the heavy (HC) and light (LC) chains were synthesized and then in each case cloned in expression vectors having a CMV promotor. They were introduced by means of lipofection into the packaging cell line (Phoenix cell line). After a 36-hour incubation, the virus supernatant was taken and used for a spinfection of the TKOs. Both the work for isolating the supernatants and the spinfection of the TKOs are widely known methods and known to a person skilled in the art.

The structural special features of subset 2 B-cell receptors were taken from the corresponding literature (see above). Exemplary CLL subset 2 VH and complete LC DNA segments were synthesized by a contract manufacturer in a standard method. They were then fused with a murine IgG1 constant segment by means of PCR and cloned in a CMV vector. The sequence of the finished vector was confirmed by means of Sanger sequencing.

CLL subset 2 VH (SEQ ID NO. 5):
EVQLVESGGGLVKPGGSLRLSCAASGFTFRSYSMNWVRQAPGKGLEWVSS
IISSSSYIYYADSVKGRFTISRDNAKNSLYLQMNSLRAEDTALYYCARDQ
NAMDVWGQGTTVTVSS
CLL-Subset 2 LC (SEQ ID NO. 6):
SYELTQPPSVSVAPGKTARITCAGNNIGSKSVHWYQQKPGQAPVLVIYYD
SDRPSGIPERFSGSNSGNTATLTISRVEAGDEADYYCQVWDSGSDHPWVF
FFTKLTVLRQPKAAPSVTLFPPSSEELQANKATLVCLISDFYPGAVTVAW
KADSSPVKAGVETTTPSKQSNNKYAASSYLSLTPEQWKSHRSYSCQVTHE
GSTVEKTVAPTECS

For the expression of the CLL subset 2 IgG1, a human cellular expression system based on HEK293T cells was used. A protocol based on polyethyleneimine (PEI) was applied for the transfection. After several passages, the supernatant was pooled and the medium contained in the combined cell supernatant was purified by means of protein G columns. The purity and quality of the soluble subset 2 IgG1 was determined by means of Western blot.

The monoclonal antibodies were produced according to the standard method in mice and with the subsequent generation of hybridoma cells. The screening for positive clones did not take place conventionally by means of ELISA. Since the target structure is a membranous receptor, it is of central importance to validate the binding of the potential antibodies also in a cellular system, i.e., by largely preserving the cell physiological states native for this cell type. First, groups of pooled supernatants were examined for binding events by means of FACS analysis. For this purpose, different CLL subset 2 BCR variants were expressed on the surface of a cell line (TKO) that cannot express an BCR itself. This way, it was at first possible to identify the supernatants whose antibodies showed a binding. Subsequently, the supernatants of the individual hybridoma clones were examined more thoroughly with respect to their binding in order to thus identify highly specific clones with high affinity.

For the screening method, different vectors were used within the scope of the preceding transformation for the following combinations of a heavy chain (HC) and a light chain (LC) of the corresponding CLL BCRs, wherein these combinations were used on the surface of the BCR reconstitution system:
Control (transformation vector without BCR) (see FIG. 1 A)
Vector with HC/LC typical for the CLL subset 2 (see FIG. 1 B)
Vector with a non-CLL subset 2 HC/an LC typical for the CLL subset 2 (without target motif; epitope) (see FIG. 1 C)
Vector with HC typical for the CLL subset 2/a non-CLL subset 2 LC (see FIG. 1 D)
Vector with a non-CLL subset 2 HC/a non-CLL subset 2 LC (see FIG. 1 E)
Vector with HC/LC typical for the CLL subset 2 (including mutation R110G (target motif)) (see FIG. 1 F).

This approach to selection is schematically shown in FIG. 1 with the example of the CLL subset 2 BCRs, wherein the designation ‘TKO’ refers to TKO cells (see above).

In the 1^{st} selection round, the supernatants of a plurality of clones were combined and examined with respect to their binding profiles on the selection matrix. A binding profile is positive if a specific binding to the “BCR of interest” is shown. Groups which showed such a profile were isolated, and the binding profile of the individual clones was once again characterized within the scope of a second selection round on the selection matrix. The binding of the monoclonal antibodies was verified using a FACS binding assay using a fluorescence-marked anti-mouse IgG antibody. The letters indicate the following: A) no BCR (control); B) a CLL subset2 typical BCR; C) a BCR with an arbitrary heavy chain and a CLL subset2 typical light chain; D) a BCR with a CLL subset2 typical heavy chain and an arbitrary light chain; E) a BCR with arbitrary heavy and light chain (control; not CLL subset2 typical BCR); F) a CLL subset2 typical BCR with a mutation in the target motif (R110G) (control).

Based on the finding that the antibody only binds to the cells with the target structures (CLL subset2 BCR; FIG. 1B), it can be concluded that in this case an antibody is present that binds specifically to cells with autonomously active receptors.

In this case, it was found that the use of cells which are in the pro-/pre-stage of B cell development is necessary for the exact expression of the BCR required for verification. These cells are in their development genetically equipped to present new BCRs by exact folding and expression on their surface. Through the inactivation (knockout) of RAG2 and lambda5, the expression of an endogenous BCR or pre-BCR is prevented. The deletion of SLP65 and the subsequent reconstruction of an inducible SLP65 makes it possible to characterize the activity level of the “BCR of interest.”

For determining the amino acid sequence of the monoclonal antibodies selected by means of selection, the mRNA was isolated from the individual hybridoma clones, and cDNA was generated therefrom, which was amplified by means of anchored PCR (Rapid expression cloning of human immunoglobulin Fab fragments for the analysis of antigen specificity of B cell lymphomas and anti-idiotype lymphoma vaccination; Osterroth F, Alkan O, Mackensen A, Lindemann A, Fisch P, Skerra A, Veelken H., J Immunol Methods 1999 Oct. 29; 229 (1-2): 141-53).

After identification and sequence determination of the regions important for the binding (CDRs), they were transferred by means of PCR to a human antibody structure. For this purpose, the VH sequence was generated in silico from the human FR regions and the murine CDR regions and subsequently synthesized as DNA fragments. They were subsequently fused by means of PCR with a human IgG1 and cloned in a vector suitable for the expression.

For generating the monoclonal antibodies, synthetic peptides, in addition to the complete immunoglobulins, were also used, which represent the regions for the capacity of an autonomous signal.

The specific monoclonal antibody against subset 2 was sequenced. In the process, the following amino acid sequences were determined, wherein the SEQ ID NO. 9 relates to the variable part of the heavy chain (HC), and the SEQ ID NO. 10 relates to the variable part of the light chain (LC), and wherein the marked regions—in the specified order—designate CDR 1, 2 and 3.

(AVA-mAb01 HC)
SEQ ID NO. 9
QVQLLQQSGPGLVQPSQSLSITCTVS [symbol] IHWVRQSPKGKGL
EWLGV [symbol] DSNAAFMSRLSITKDNSKSQVFFKMNSLQADDTAI
YYC [symbol] WGQGTSVTVSS
(AVA-mAb01 LC)
SEQ ID NO. 10
QIVLTQSPASLSASVGETVTITCRAS [symbol] LAWYQQKQGKSPQLLV
Y [symbol] TLADGVPSRFSGSGSGTQYSLKINSLQPEDFGSYYC [symbol]
FGAGTKLELK

The partial sequences of the heavy chain corresponding to CDR1, CDR2 and CDR3 according to SEQ ID NO. 9 are specified in SEQ ID NOS. 11 to 13, while the partial sequences of the light chain corresponding to CDR1, CDR2 and CDR3 according to SEQ ID NO. 10 are shown in SEQ ID NOS. 14 to 16.

(AVA-mABO1 CDR1 HC)
SEQ ID NO. 11
GFSLTSYG
(AVA-mABO1 CDR2 HC)
SEQ ID NO. 12
IWRGGGT
(AVA-mABO1 CDR3 HC)
SEQ ID NO. 13
ARSRYDEEESMNY
(AVA-mABO1 CDR1 LC)
SEQ ID NO. 14
GNIHSY
(AVA-mABO1 CDR2 LC)
SEQ ID NO. 15
NAKT
(AVA-mABO1 CDR3 LC)
SEQ ID NO. 16
QHFWNTPPT

The above-described approach is exemplary for the generation of specific antibodies with respect to CLL subset 2. The same process was also carried out using specific sequences and isotypes for subset 4.

Exemplary CLL subset 4 VH and complete LC DNA segments were synthesized by a contract manufacturer in a standard method. They were then fused with a murine IgG1 constant segment by means of PCR and cloned in a CMV vector. The sequence of the finished vector was confirmed by means of Sanger sequencing.

CLL subset 4 HC (SEQ ID NO. 7):
QVQLQQWGAGLLKPSETLSLTCAVYGGSFSGYYWTWIRQSPGKGLEWIGE
INHSGSTTYNPSLKSRVTISVDTSKNQFSLKLNSVTAADTAVYYCARGYG
DT [symbol] MDVWGQGTTVTVSSASTKGPSVFPLAPSSKSTSGGTAA
LGCLVKDYFPEPVTVSWNSGALTSGVHTFPACLQSSGLYSLSSVVTVPSS
SLGTQTYICNV [symbol] DKKC

The regions in bold type denote the target sequences (epitopes) of the variable part of the heavy chain of the BCR of subset 4, which are responsible for its autonomously active state (cf. SEQ ID NOS. 3 and 4).

CLL subset 4 LC (SEQ ID NO. 8):
DIVMTQSPLSLPVTLGQPASISCRSSQSLVHSDGNTYLNWFQQRPGQSPR
RLIYKVSDRDSGVPDRFSGSGSGTDFTLKISRVEAEDVGLYYCMQGTHWP
PTFGQGTKVEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC.

1. A method for selecting a biological binding molecule that specifically binds to an autonomously active or autonomously activated B-cell receptor as target receptor, but not to a non-active or non-activated B-cell receptor, within the scope of a cell-based system using immature B cells in the pro-/pre-stage, comprising the following steps:
a. providing a plurality of biological binding molecules obtained through immunization of a mammal with B-cell receptors or their fragments and subsequent immortalization and purification;
b. providing immature B cells in the pro-/pre-stage, which are not capable of expressing the native genes for RAG2 and/or RAG1 and lambda5 but which were enabled to express autonomously active or autonomously activated B-cell receptors as target receptors on their cell surface;
c. providing immature B cells in the pro-/pre-stage, which are not capable of expressing the native genes for RAG2 and/or RAG1 and lambda5 but which were enabled to express non-active or non-activated B-cell receptors as reference receptors on their cell surface;
d. comparatively examining the binding behavior of the binding molecules provided according to step (a) with respect to cells provided according to steps (b) and (c);
e. selecting at least one binding molecule that binds specifically to cells provided according to step (b) but not to cells provided according to step (c).

2. The method according to claim 1, characterized in that the cells provided according to step (b) are also not capable of expressing the native gene SLP65.

3. The method according to claim 1, characterized in that cells are provided according to step (c), which express a non-autonomously active B-cell receptor as a reference receptor.

4. The method according to claim 2, characterized in that, in addition to determining a specific binding of the binding molecule to cells provided according to step (b), step (e) includes a confirmation through an activity measurement after the induction of SLP65.

5. The method according to claim 2, characterized in that cells are provided according to step (c), which express a non-autonomously active B-cell receptor as a reference receptor.

6. The method according to claim 3, characterized in that, in addition to determining a specific binding of the binding molecule to cells provided according to step (b), step (e) includes a confirmation through an activity measurement after the induction of SLP65.

7. The method according to claim 3, characterized in that the cells provided according to step (b) are also not capable of expressing the native gene SLP65 and in addition to determining a specific binding of the binding molecule to cells provided according to step (b), step (e) includes a confirmation through an activity measurement after the induction of SLP65.
